(19) United States
(12) Patent Application Publication
(10) Pub. No.: US 2022/0047902 A1
(43) Pub. Date: Feb. 17, 2022

(54) AEROSOL-REDUCING VACUUM FACEMASK

(71) Applicant: UNIVERSITY OF UTAH RESEARCH FOUNDATION, Salt Lake City, UT (US)

(72) Inventors: Bryan R. McRae, Salt Lake City, UT (US); Jared L. Smith, Salt Lake City, UT (US)

(21) Appl. No.: 17/400,008

(22) Filed: Aug. 11, 2021

Related U.S. Application Data
(60) Provisional application No. 63/064,226, filed on Aug. 11, 2020.

(51) Int. Cl. A62B 23/02; A62B 18/08; A41D 13/11

(52) U.S. Cl. CPC A62B 23/025; A41D 13/11; A62B 18/084

(57) ABSTRACT

Improved facemask designs are disclosed herein. The improved facemask is designed to provide a sucking airflow that sucks aerosolized particles emitted from a person's mouth towards a filtration unit. By providing this sucking airflow, medical personnel can work on the user's mouth and facial area without worrying about breathing in the aerosolized particles. The facemask includes a covering portion that covers a user's nose and mouth. The facemask includes a central lumen that is configured to permit access to the user's nose and/or mouth. The facemask further includes an access port configured to enable airflow communication between the covering portion and a bilateral vacuum. Airflow is directed away from the central lumen towards the bilateral vacuum. By directing the airflow in this manner, aerosolized particles emitted from the user's mouth are removed in an effective amount to decrease expulsion of the aerosolized particles outside of the vacuum facemask.

### CROSS-REFERENCE TO RELATED APPLICATIONS

This application claims priority to U.S. Provisional Application Ser. No. 63/064,226, filed on Aug. 11, 2020, and entitled “AEROSOL-REDUCING VACUUM FACEMASK,” the entirety of which is incorporated herein by reference.

### BACKGROUND

SARS-CoV-2 is the virus that caused the worldwide pandemic known as COVID-19 (“CO” stands for “corona,” “VI” stands for “virus,” “D” stands for “disease,” and “19” refers to the year 2019 or the year in which the virus originated). COVID-19 has impacted almost every person across the entire globe and has results in hundreds of thousands of deaths worldwide.

COVID-19 is a respiratory virus that spreads via emission of droplets projected from a person's mouth and nose. COVID-19 can cause respiratory illness and other complications, such as inflammation, pneumonia, and others.

A hotly debated topic is whether facemasks can be used to help stop or slow the spread of SARS-CoV-2. A facemask is a type of material that covers a person's nose and mouth. There are many different types of facemasks currently in use, ranging from cloth masks to surgical masks to N95 masks to respirators. Although facemasks have been around for almost all time, there is still a need to provide an improved type of facemask, especially during the time of SARS-CoV-2.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

Embodiments disclosed herein relate to an improved type of facemask or face covering. In particular, the improved facemask is designed to provide a sucking airflow that sucks aerosolized particles emitted from a person's mouth or nose towards a filtration unit. By providing this sucking airflow, medical personnel can work on the user's mouth and facial area without worrying about breathing in the aerosolized particles.

In some embodiments, a vacuum facemask includes a covering portion that is configured to cover at least a nose and a mouth of a user. The facemask also includes a central lumen positioned in the covering portion. The central lumen is configured to permit access to the nose and/or the mouth of the user. The facemask further includes an access port configured to enable airflow communication between the covering portion and a bilateral vacuum, which provides suction to generate an airflow. A vacuum tube connects the bilateral vacuum to the access port of the facemask, and the airflow is directed away from the central lumen towards the bilateral vacuum via the vacuum tube. By directing the airflow in this manner, the embodiments reduce or remove aerosolized particles emitted from the user's mouth and nose in an effective amount to decrease expulsion of the aerosolized particles to regions outside of the vacuum facemask.

In some embodiments, a vacuum facemask comprises a covering portion that is configured to cover a portion of a face of user and a central lumen positioned in the covering portion. The facemask also includes a first access port and a second access port. The first and second access ports are configured to enable communication with a vacuum. Padding is disposed on a rear surface of the vacuum facemask. This padding is positioned on the vacuum facemask at a position where the vacuum facemask contacts the user's face. A first portion of a vacuum tube connects the vacuum to the first access port, and a second portion of the vacuum tube connects the vacuum to the second access port. The vacuum provides a sucking airflow, which sucks air away from the central lumen towards the first access port and towards the second access port.

In some embodiments, a vacuum facemask comprises a covering portion that is configured to cover a portion of a face of user and a central lumen positioned in the covering portion. The facemask also includes a first access port and a second access port. The first and second access ports are configured to enable communication with a vacuum. Padding is disposed on a rear surface of the vacuum facemask. The padding is positioned on the vacuum facemask at a position where the vacuum facemask contacts the user's face. The facemask also includes a first pair of strap anchors and a first flexible strap that is coupled to the first pair of strap anchors and that is configured to wrap around a head of the user. The facemask further includes a second pair of strap anchors and a second flexible strap that is coupled to the second pair of strap anchors and that is configured to wrap around the user's head. A vacuum tube connects the vacuum to at least one of the first access port or the second access port. The vacuum provides a sucking airflow, which sucks air away from the central lumen towards the first access port or the second access port.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates how an improved type of vacuum facemask can be work by a user.

FIG. 2 illustrates how straps of the vacuum facemask can be used to affix the facemask to a user.

FIGS. 3A and 3B illustrate how the facemask can include vacuum tubing and can be coupled to a vacuum in order to suck up droplets that occur when a user breathes.

FIG. 4A illustrates a front perspective view of the facemask.

FIG. 4B illustrates an angled side perspective view of the facemask.

FIG. 4C illustrates a side perspective view of the facemask.

FIG. 4D illustrates an angled rear perspective view of the facemask.

FIG. 4E illustrates a rear perspective view of the facemask.

FIG. 4F illustrates an angled rear perspective view of the facemask.

FIG. 4G illustrates an angled side perspective view of the facemask.

FIG. 4H illustrates an angled side perspective view of the facemask.

FIG. 4I illustrates a flat perspective view of the facemask.

FIG. 4J illustrates an angled side perspective view of the facemask.

FIG. 4K illustrates an angled side perspective view of the facemask.

FIG. 4L illustrates a side perspective view of the facemask.

FIG. 4M illustrates an angled side perspective view of the facemask.

FIG. 5 illustrates various angled perspective views of the facemask.

### DETAILED DESCRIPTION

Embodiments disclosed herein relate to an improved type of facemask or face covering. In particular, the improved facemask is designed to provide a sucking airflow that sucks aerosolized particles emitted from a person's mouth towards a filtration unit. By providing this sucking airflow, medical personnel can work on the user's mouth and facial area without worrying about breathing in the aerosolized particles.

In some embodiments, a vacuum facemask includes a covering portion that is configured to cover at least a user's nose and mouth. The facemask also includes a central lumen positioned in the covering portion. The central lumen permits access to the nose and/or mouth of the user. The facemask further includes an access port configured to enable airflow communication between the covering portion and a bilateral vacuum, which provides suction to generate airflow. A vacuum tube connects the bilateral vacuum to the access port of the facemask, and the airflow is directed away from the central lumen towards the bilateral vacuum via the vacuum tube. By directing the airflow in this manner, the embodiments reduce or remove aerosolized particles emitted from the user's mouth in an effective amount to decrease expulsion of the aerosolized particles to regions outside of the vacuum facemask.

In some embodiments, a vacuum facemask comprises a covering portion that is configured to cover a portion of a user's face and further includes a central lumen positioned in the covering portion. The facemask also includes a first access port and a second access port. The first and second access ports are configured to enable communication with a vacuum. Padding is disposed on a rear surface of the vacuum facemask. This padding is positioned on the vacuum facemask at a position where the vacuum facemask contacts the user's face. A first portion of a vacuum tube connects the vacuum to the first access port, and a second portion of the vacuum tube connects the vacuum to the second access port. The vacuum provides a sucking airflow, which sucks air away from the central lumen towards the first access port and towards the second access port.

In some embodiments, a vacuum facemask comprises a covering portion that is configured to cover a portion of a face of user and a central lumen positioned in the covering portion. The facemask also includes a first access port and a second access port. The first and second access ports are configured to enable communication with a vacuum. Padding is disposed on a rear surface of the vacuum facemask. The padding is positioned on the vacuum facemask at a position where the vacuum facemask contacts the user's face. The facemask also includes a first pair of strap anchors and a first flexible strap that is coupled to the first pair of strap anchors and that is configured to wrap around a head of the user. The facemask further includes a second pair of strap anchors and a second flexible strap that is coupled to the second pair of strap anchors and that is configured to wrap around the user's head. A vacuum tube connects the vacuum to at least one of the first access port or the second access port. The vacuum provides a sucking airflow, which sucks air away from the central lumen towards the first or second access ports.

### Examples of Technical Benefits & Improvements

The following section outlines some example improvements and practical applications provided by the disclosed embodiments. It will be appreciated, however, that these are just examples only and that the embodiments are not limited to only these improvements.

The disclosed technology relates to an aerosol reducing vacuum facemask that is worn by a patient who may be asymptomatic or symptomatic with communicable respiratory illness, such as perhaps SARS-CoV-2. It is anticipated that this facemask could help to prevent, mitigate, or otherwise reduce droplets and saliva from being aerosolized into the environment during aerosol-generating procedures involving the upper aerodigestive tract, such as during a dental, otolaryngologic/ENT, or throat swab procedure. This developed facemask helps to remove aerosolize particles through vacuum suction at the entry of the nose and mouth, thereby helping to decrease the risk of infection to health care providers when providing care to the patient, even those patients who have SARS-CoV-2.

Currently, there are many different masks that health care providers can wear to prevent exposure to communicable diseases, such as SARS-CoV-2, in a healthcare setting. Such masks include a filtering facepiece respirator (FFR), elastomeric half facepiece respirator, elastomeric full facepiece respirator, and a powered air-purifying respirator (PAPR). These different types of masks help protect healthcare workers while they provide care to patients; however, there is still a high risk of infection due to aerosols that can be released by patients undergoing medical procedures. Patients are usually provided with a surgical mask when receiving oxygen through a nasal cannula or through oxygen devices such as a tracheostomy to help protect health care workers. While the above-described masks are readily available, there is still risk of aerosolized particle exposure to healthcare workers. What is needed, therefore, is improved devices or technologies that reduce patient-generated aerosols from the patient's perspective when receiving care.

The COVID-19 pandemic has increased the safety risks from respiratory droplets and aerosols during common procedures (e.g., dental, otolaryngologic/ENT, throat swabs, etc.), and healthcare worker infections persist. The disclosed embodiments effectively contain and reduce oral and nasal droplets and upper aerodigestive tract aerosols during medical procedures or examinations, while still optionally providing open access for medical or dental instruments.

The disclosed embodiments beneficially provide adequate vacuum strength and containment designed to forcefully direct or propagate exhaled droplets or aerosols through certain defined “access ports” disposed on the facemask, thereby preventing those droplets from being breathed in by medical personnel examining the person wearing the facemask. Current solutions either focus on gas containment (mercury amalgam) or on suction/vacuum of dental irrigation liquids.

Effective vacuum mask devices (such as those disclosed herein) beneficially reduce healthcare workers' risks of infection from aerosolized or droplet particles from patients. The embodiments also beneficially reduce risks of infections spreading among multiple patients present in an office setting. The reduced droplet/aerosol risk could also permit reduced use of PPE equipment or could augment the efficacy of the PPE already being worn.

### Vacuum Facemask Positions on a User

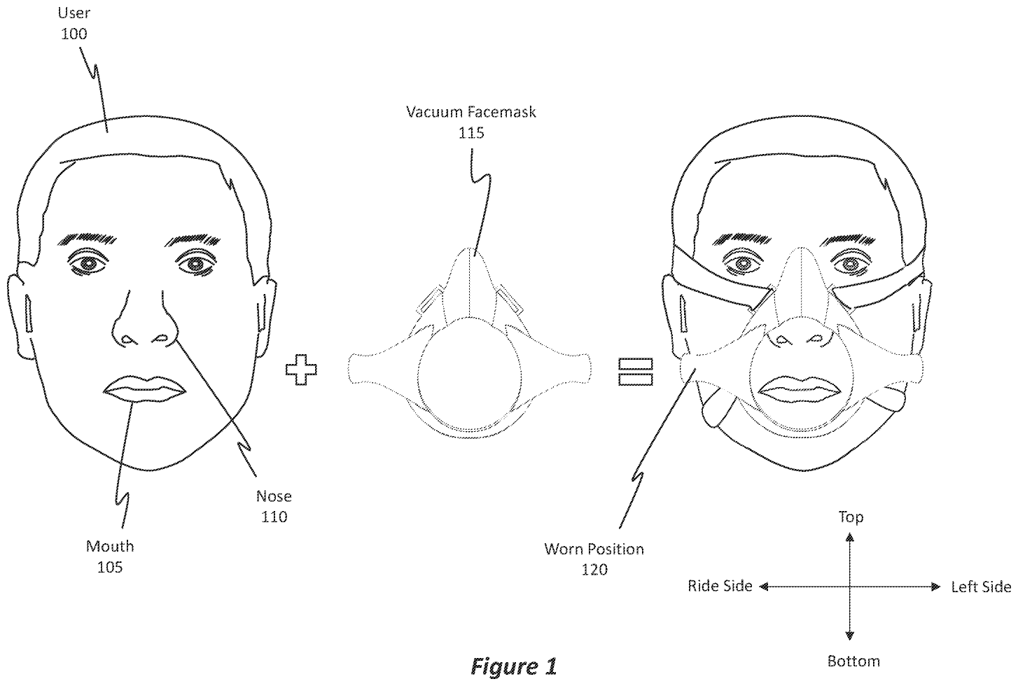

Attention will now be directed to FIG. 1, which illustrates a user 100 who has a mouth 105 and a nose 110. FIG. 1 also shows an example of a vacuum facemask 115 designed in accordance with the disclosed principles, which will be discussed in more detail shortly. The vacuum facemask 115 is designed to be worn by the user 100 in the manner shown by the worn position 120. Specifically, the worn position 120 shows how the vacuum facemask 115 covers the user's mouth 105 and nose 110. In some implementations (as will be discussed shortly), the vacuum facemask provides a hollowed opening to permit a medical professional to examine and operate on the mouth 105 and the nose 110 or other facial features. For instance, FIG. 1 shows how the user's mouth and nose are visible and accessible via the hollowed portion of the vacuum facemask (e.g., the mouth and nose are visible in the right-hand illustration of FIG. 1).

FIG. 1 also shows a direction legend. Specifically, the legend calls out the top side of the vacuum facemask 115. The legend also shows the bottom, left, and right sides. The remaining figures will follow the same directional scheme outlined by the legend provided in FIG. 1.

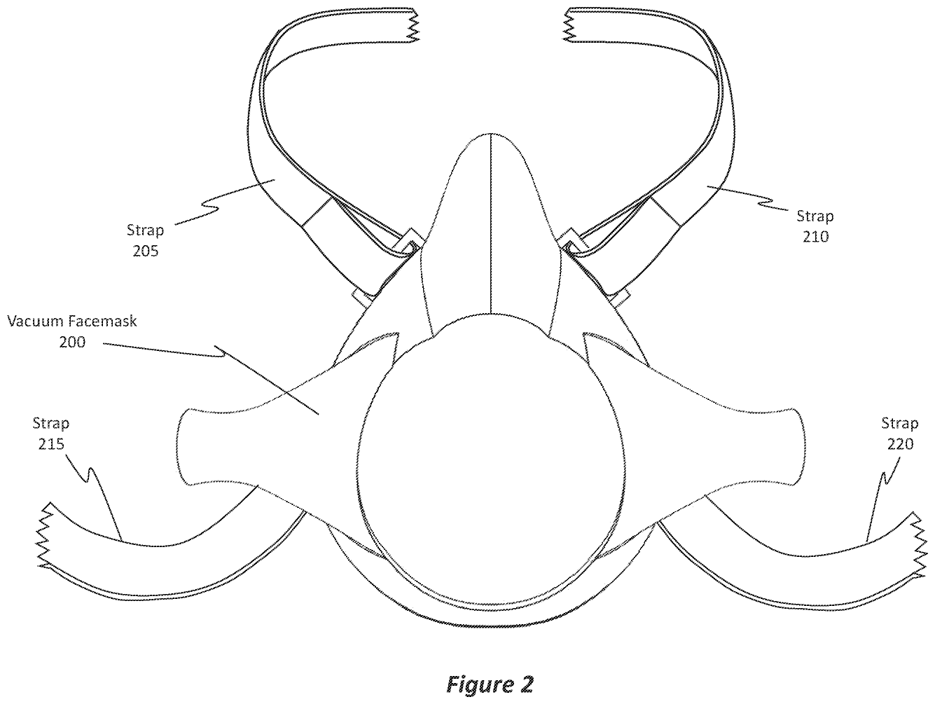

FIG. 2 shows a vacuum facemask 200, which is representative of the vacuum facemask 115 of FIG. 1. In some embodiments, the vacuum facemask 200 includes a first flexible strap, which is shown as strap 205 and strap 210. Straps 205 and 210 can be a part of the same strap. FIG. 2 shows a diagonal cut between the straps 205 and 210 to illustrate how the flexible strap can flex and stretch to different sizes. In any event, the first flexible strap can be coupled to the vacuum facemask 200 via a pair or set of strap anchors (to be discussed in more detail later) and can be wrapped around the user's head.

The vacuum facemask 200 is further shown as including a second flexible strap, which is shown as strap 215 and strap 220. Straps 215 and 220 can be a part of the same strap. FIG. 2 shows a diagonal cut between straps 215 and 220 to illustrate how the flexible strap can flex and stretch to different sizes in order to accommodate different head sizes. That is, the second flexible strap can be coupled to the vacuum facemask 200 via a second pair or set of strap anchors and can be wrapped around the user's head.

The flexible straps can be made from any type of flexible material that is suitable for contact with a person's head. Example materials include, but are not limited to, bungee cords, rubber bands, cloth, Velcro, or any other flexible material. Using the flexible straps, the user is able to secure the vacuum facemask 200 to his/her own face.

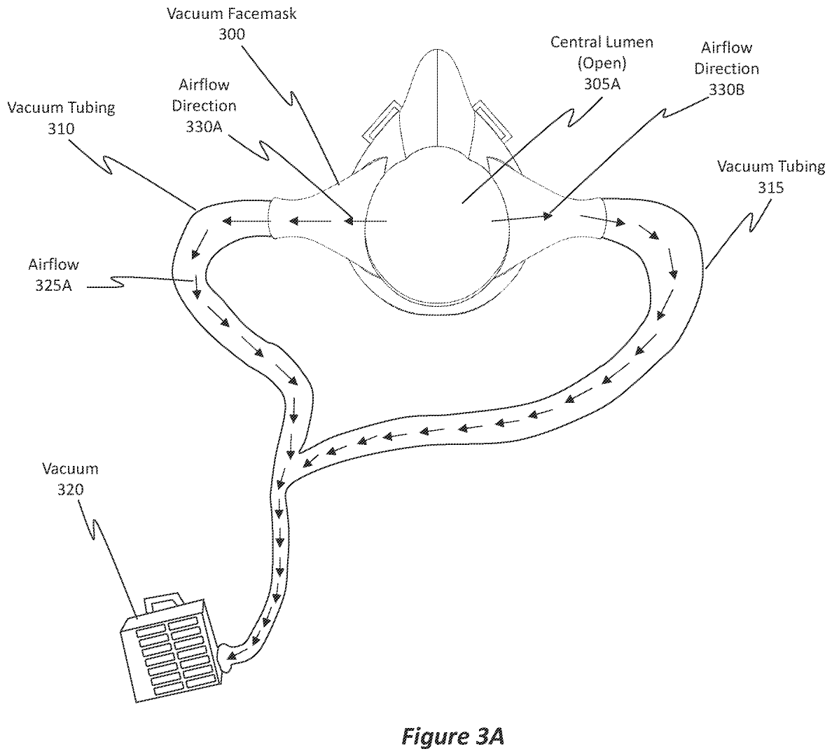

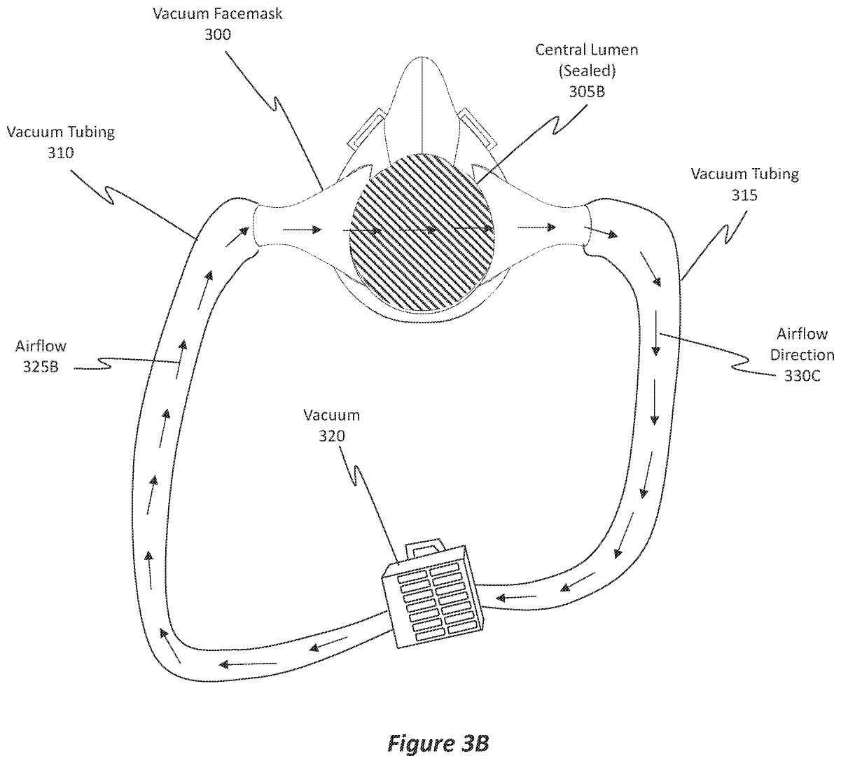

FIGS. 3A and 3B show different features of the disclosed embodiments. Beneficially, the embodiments are able to suck air away from a user's mouth in an effort to capture, contain, or redirect emitted aerosol particles towards a desired direction as opposed to being sprayed outward or beyond the regions confined by the facemask.

FIG. 3A shows one example implementation for sucking air away from a user's mouth. Specifically, FIG. 3A shows a vacuum facemask 300, which is representative of the vacuum facemask 200 of FIG. 2. The vacuum facemask 300 includes a central lumen 305A, which is an open or hollowed area of the vacuum facemask 300 and which permits access to the user's mouth and nose.

The vacuum facemask 300 also includes vacuum tubing 310 and vacuum tubing 315. These tubing materials are coupled to the vacuum facemask 300 at certain access ports of the vacuum facemask (the access ports will be discussed in more detail momentarily). The tubing can be comprised of any type of flexible material designed to control airflow. Example materials include any type of plastic or rubber, among others.

The vacuum facemask 300 is a part of a unit that further includes a vacuum 320, such as a bidirectional vacuum. The vacuum 320 can include a filter designed to filter out contaminants from an airstream, including viral contaminants or other disease contaminants.

Notice, in FIG. 3A, the vacuum tubing 310 and the vacuum tubing 315 are different portions of the same tubing. That is, the tubing is initially coupled to the vacuum 320 and then splits into two parts, with one part connecting to a first side of the vacuum facemask 300 and another part connecting to a second side of the vacuum facemask 300. With this configuration, the airflow 325A is directed from the facemask 300 towards the vacuum 320. For instance, FIG. 3A shows a first airflow direction 330A, which is a direction originating from the central lumen 305A outwards towards the vacuum tubing 310. FIG. 3A shows a second airflow direction 330B, which is a direction also originating from the central lumen 305A but is directed outwards towards the vacuum tubing 315.

In some embodiments, the airflow direction 330B is opposite the airflow direction 330A. In some embodiments, depending on the direction of the access ports, the two airflow directions 330A and 330B are at least 90 degrees opposite one another. In some embodiments, the two airflow directions 330A and 330B are anywhere between 90 degrees and 180 degrees opposite one another. In any event, the embodiment shown in FIG. 3A is useful for scenarios where access to the user's mouth and nose is desirable.

FIG. 3B, on the other hand, illustrates a scenario where access to the user's mouth and nose are not necessary. Specifically, FIG. 3B shows similar features as those presented in FIG. 3A. Now, however, the central lumen 305B is or includes a sealed portion of the vacuum facemask 300. To clarify, whereas the central lumen 305A in FIG. 3A was a hollowed out portion of the vacuum facemask 300, the central lumen 305B in FIG. 3B is not hollowed out but rather includes a material that seals the vacuum facemask 300 at that region or area. In this configuration, the material forming or covering the central lumen 305B can be transparent or opaque. With this configuration, the vacuum 320 can be configured to provide the airflow 325B, and that airflow 325B can have flow in a single circulating direction, as shown by airflow direction 330C. That is, the airflow 325B can flow into the vacuum facemask 300 via the vacuum tubing 310, across the user's face, and then can exit the vacuum facemask 300 via the vacuum tubing 315, thereby removing particles and aerosolized droplets. Such a configuration is beneficial in environments where a protective facemask is required.

### Vacuum Facemask Configurations

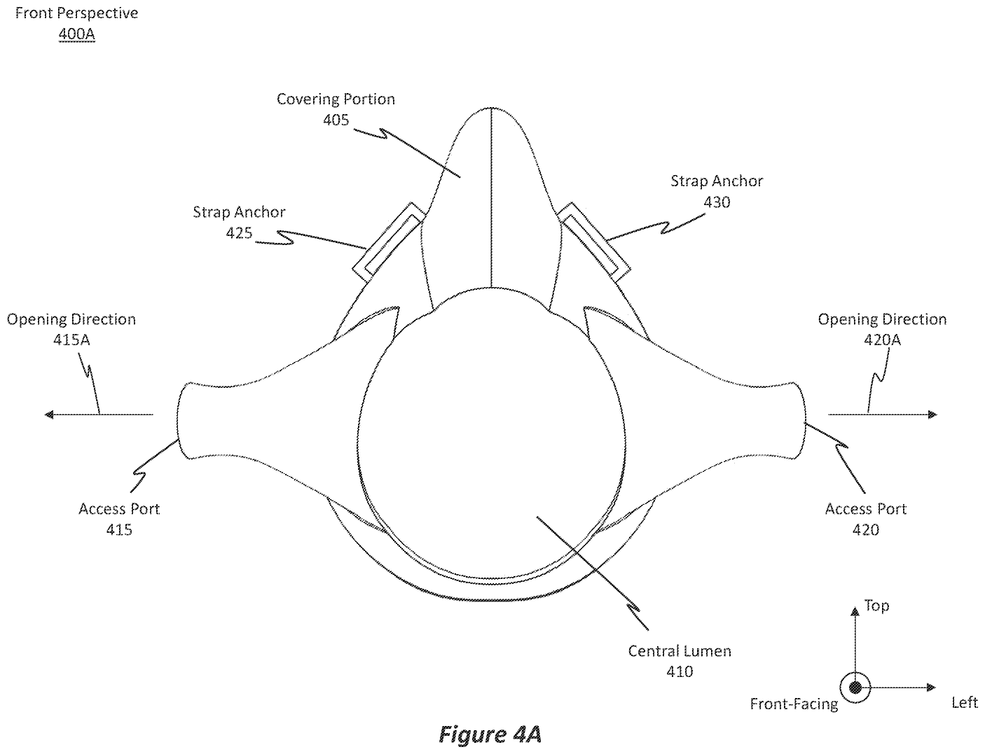

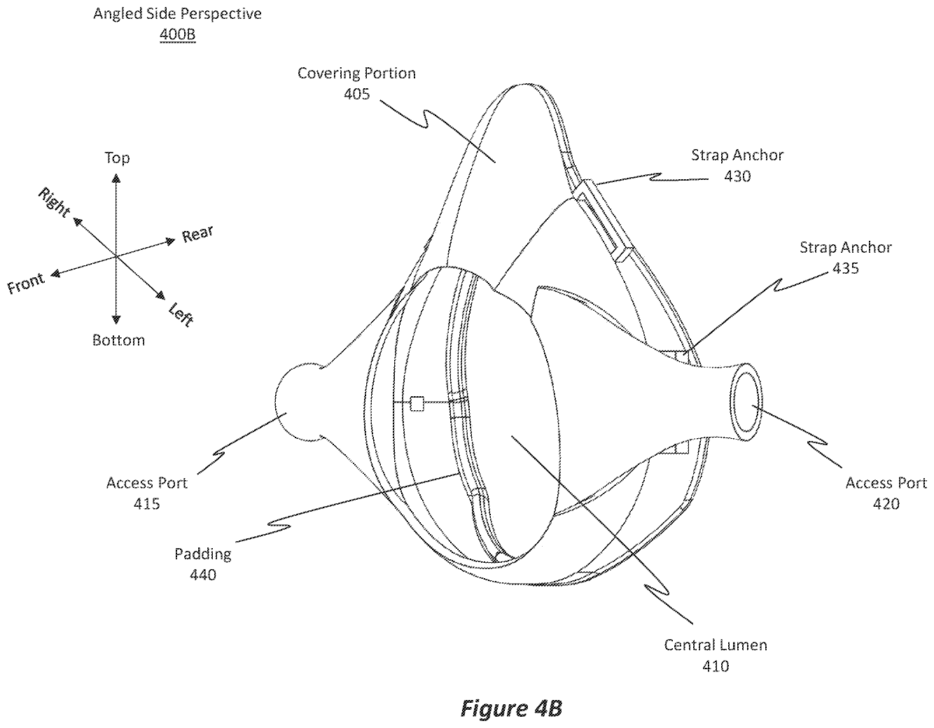

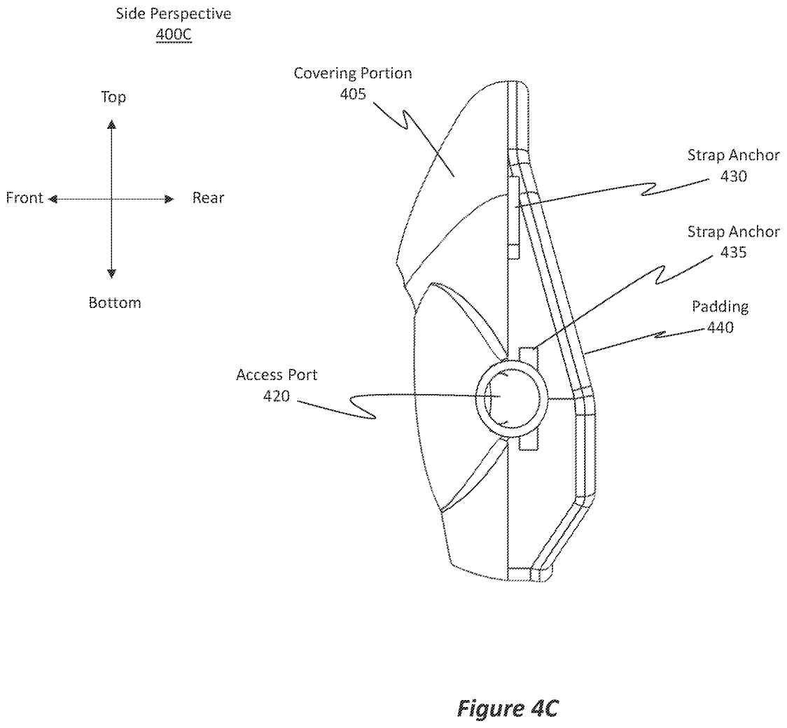

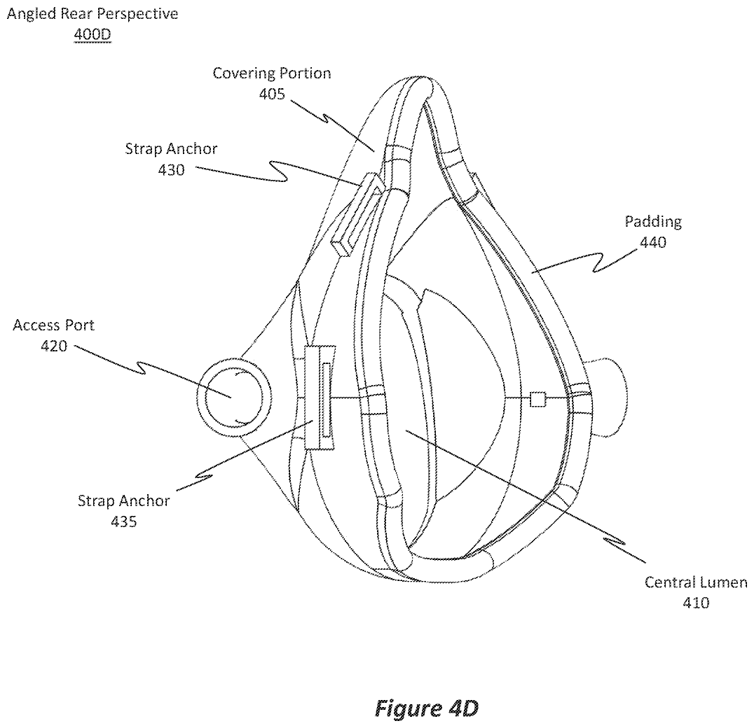

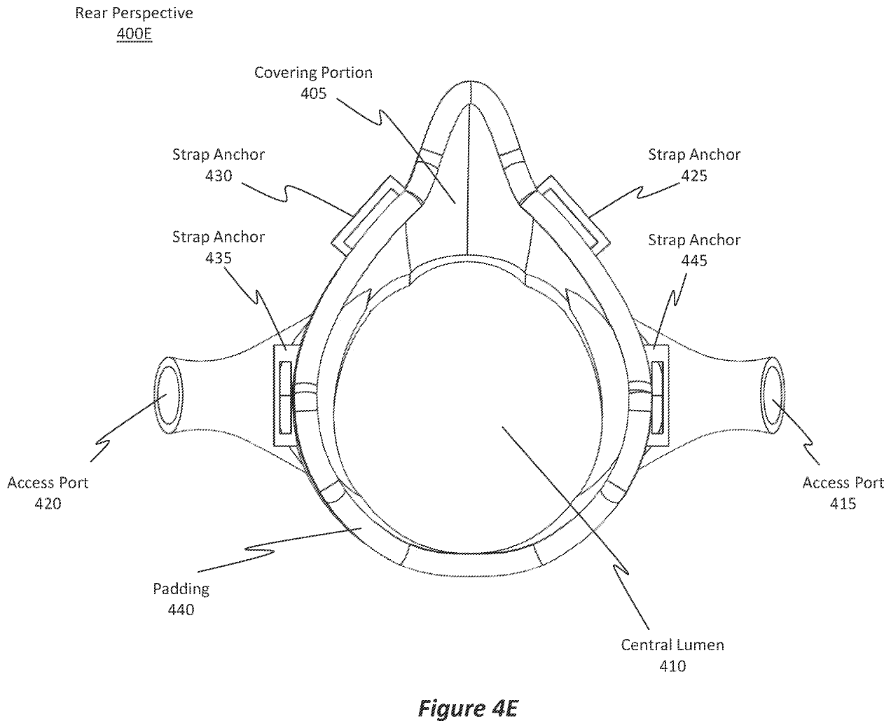

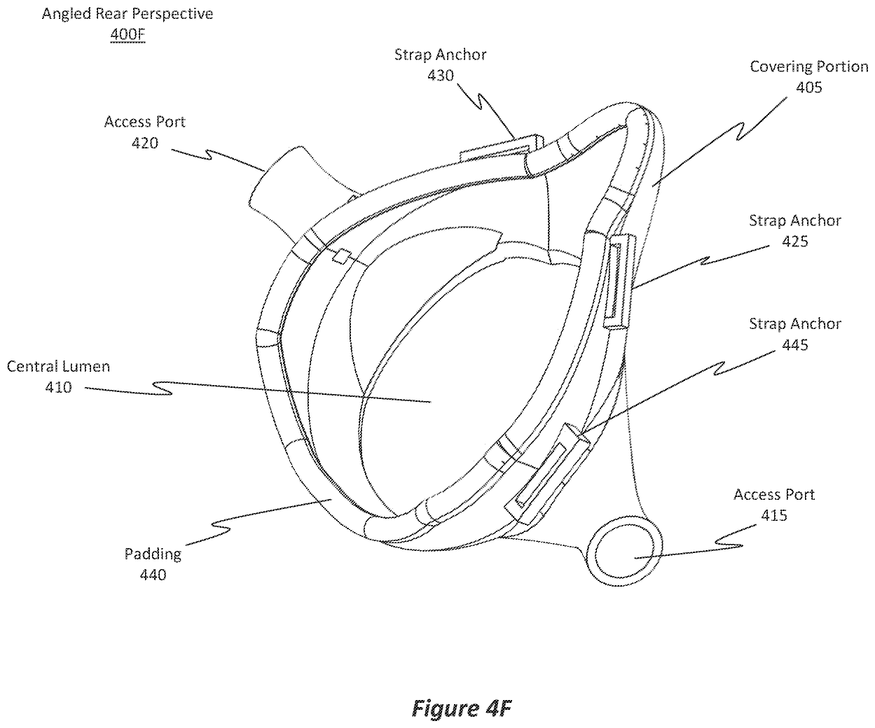

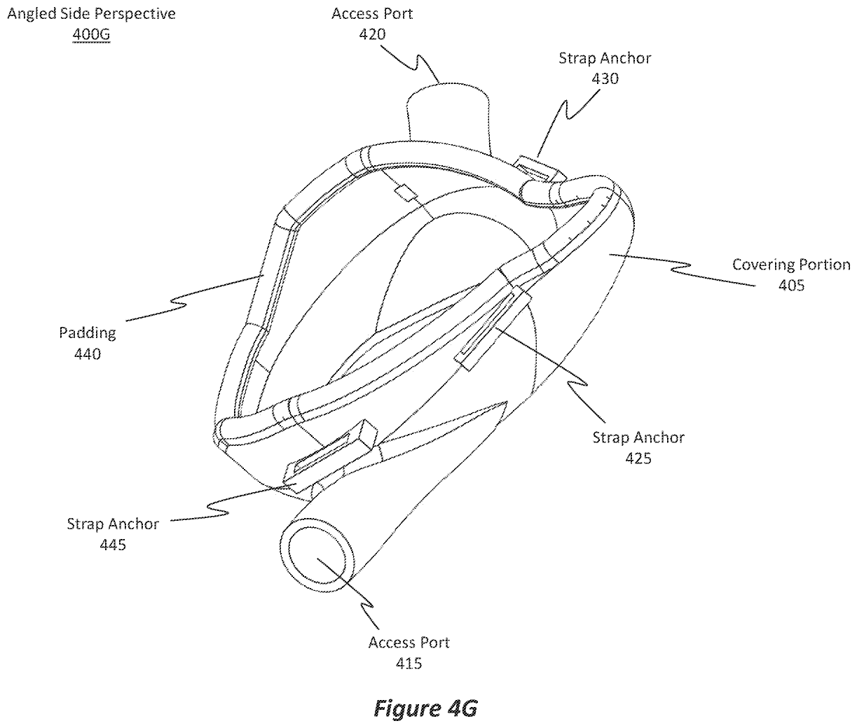

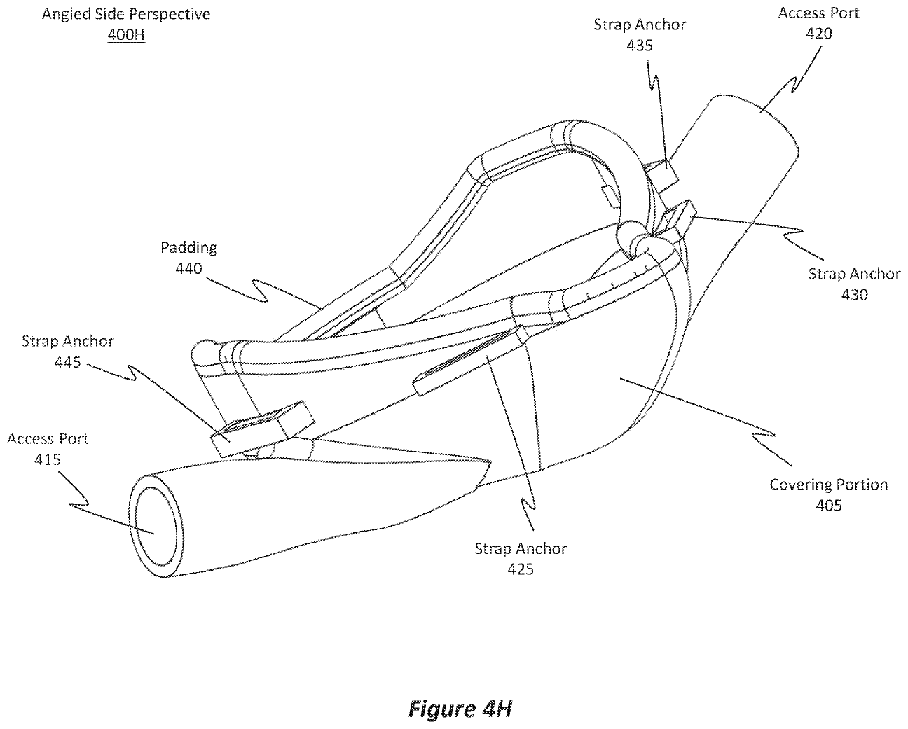

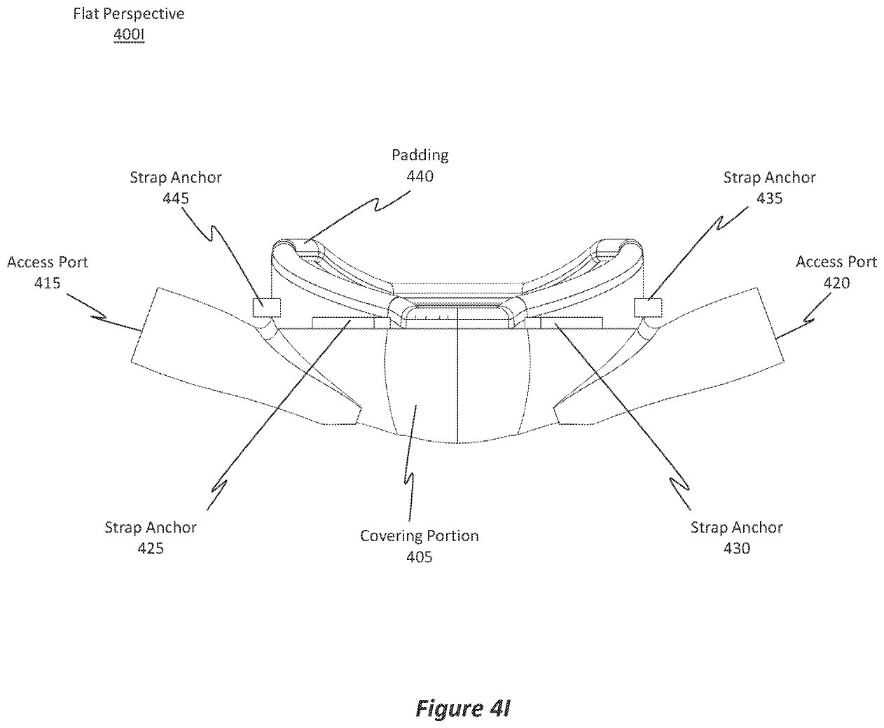

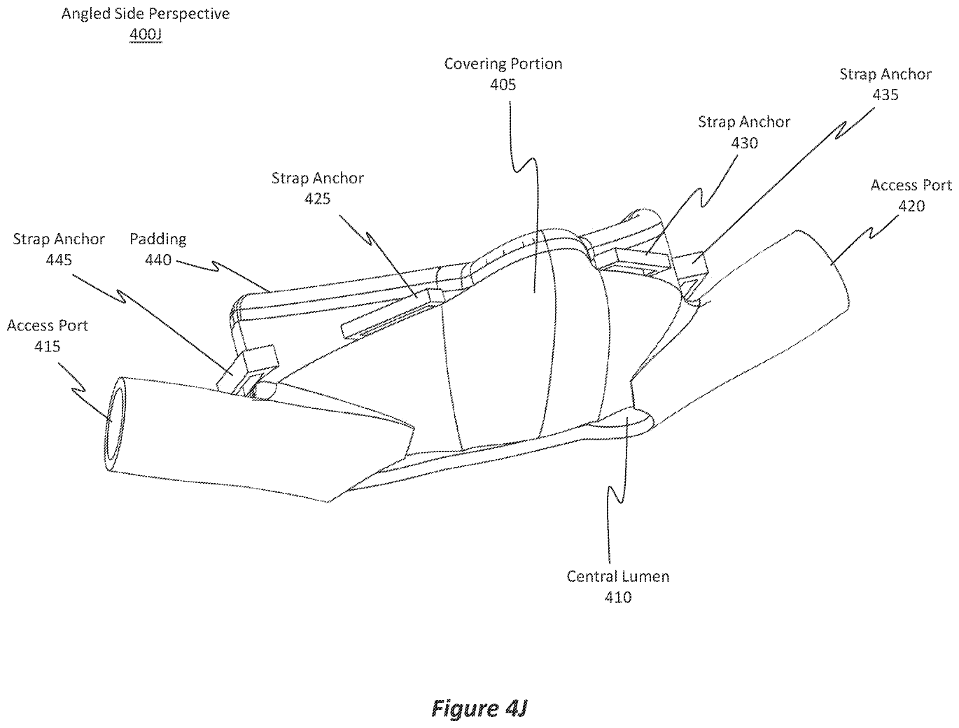

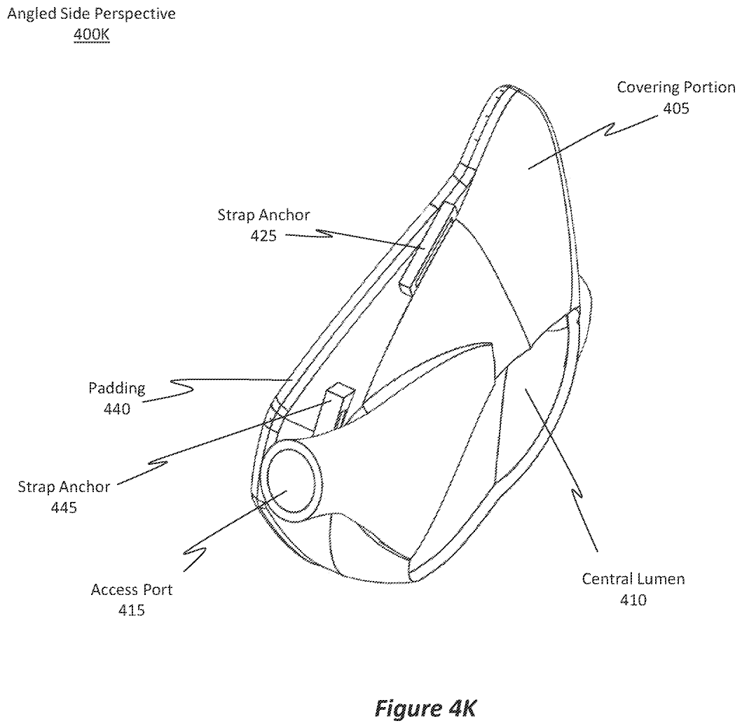

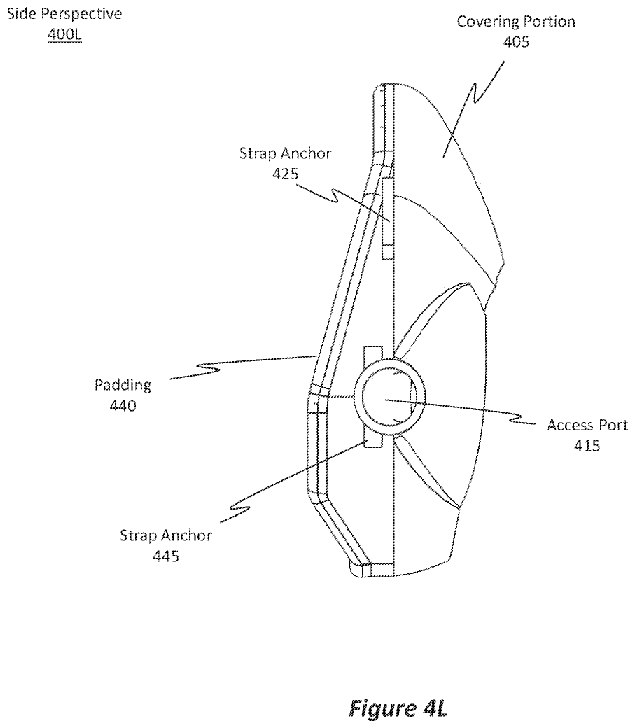

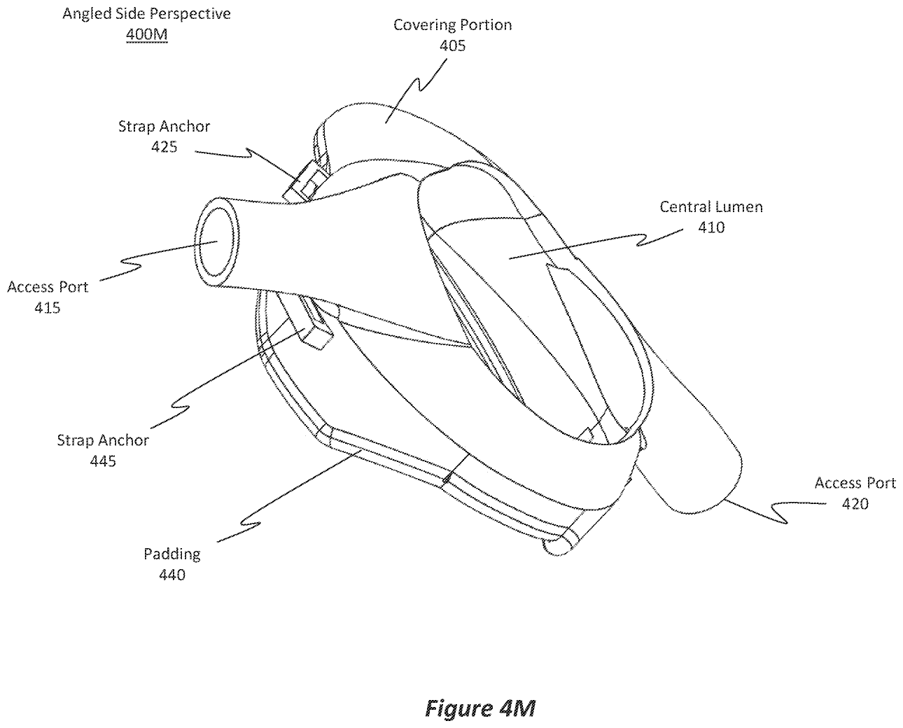

FIGS. 4A through 4G illustrate various different perspectives and views of a vacuum facemask, such as those discussed in FIGS. 1 through 3B. Common labels have been used throughout these figures to illustrate the common features of the vacuum facemask.

FIG. 4A shows a front perspective 400A of the disclosed vacuum facemask. The vacuum facemask includes a covering portion 405, which is configured to cover at least a nose and a mouth of a user. In some embodiments, the facemask utilizes adapter, full-face snorkel masks, and adapter anesthesia masks as the covering portion 405. Of course, the covering portion 405 can be made of any material that is capable of filtering or at least restricting the flow of vapor droplets. For instance, the covering portion 405 can be comprised of plastic, metal, rubber, or even stiff cloth or fiber material.

The vacuum facemask also includes a central lumen 410, which is positioned on the covering portion 405 and which is configured to permit access to the nose and/or the mouth of the user. In some implementations, the central lumen 410 is a hollowed out portion of the covering portion 405. In some cases, the central lumen 410 is comprised of a removable material that is transparent or opaque. The removable material can be affixed to the covering portion 405 via a snap connection, a hinge mechanism, or another other clasp mechanism.

The vacuum facemask includes a first access port 415 that is configured to enable airflow communication between the covering portion 405 and a single, bilateral, or multi-port vacuum, such as the vacuum 320 of FIGS. 3A and 3B. Notice, the access port 415 has an opening direction 415A where air can exit or enter the vacuum facemask.

The vacuum facemask also includes a second access port 420 that is configured to enable airflow communication between the covering portion 405 and the bilateral vacuum, which provides suction to generate an airflow. Notice, the access port 420 has an opening direction 420A. In some cases, the opening direction 420A is opposite the opening direction 415A (e.g., they are 180 degrees opposite relative to one another). In some implementations, the opening direction 420A is less than 180 degrees opposite the opening direction 415A but is greater than 90 degrees. In this sense, the relativity between the opening direction 420A and 415A can be anywhere between about 90 degrees and 180 degrees.

The vacuum facemask is also shown as including a first pair of strap anchors, which pair includes the strap anchor 425 and 430. Although not shown in FIG. 4A, later figures will show how the vacuum facemask can include a second pair of strap anchors. The first pair of strap anchors can be used to connect the first flexible strap (comprising strap 205 and 210 from FIG. 2) to the vacuum facemask. The second pair of strap anchors can be used to connect the second flexible strap (comprising strap 215 and 220 from FIG. 2) to the vacuum facemask as well.

FIG. 4A also shows a direction legend, which includes a top direction, a left direction, and a front-facing direction (i.e. extending out of the paper towards the reader). The front-facing direction represents the front of the vacuum facemask. The legend follows the same directional arrangement as the legend presented in FIG. 1.

The vacuum tubing mentioned earlier is connected to the access port 415 and 420. In this manner, the vacuum tube can connect the bilateral vacuum to the access port(s) of the facemask. With this configuration, airflow (which is provided by suction generated by the vacuum) can be directed away from the central lumen 410 towards the bilateral vacuum via the vacuum tube to reduce or remove aerosolized particles emitted from a mouth of the user in an effective amount to decrease expulsion of the aerosolized particles from areas or regions outside of the vacuum facemask. In some cases, the embodiments include a single vacuum tube that is split such that the vacuum tube not only connects to a first one of the access ports but it additionally connects the second access port to the bilateral vacuum.

Notice, in FIG. 4A, the access port 415 is disposed on a first lateral position (e.g., the right-hand position, as defined by the legend) of the vacuum facemask. The second access port (e.g., access port 420) is disposed on a second lateral position (e.g., the left-hand position, as defined by the legend) of the vacuum facemask. Such positioning can be modified, however. For instance, the positioning of the access ports 415 and 420 can be more closely proximate to the central lumen 410 and can optionally be angled or directed more towards the front-facing direction, whereas currently they are illustrated as being angled outward towards the left and right. For instance, in some embodiments, the opening direction 415A of the access port 415 is orthogonal to a front-facing direction of the vacuum facemask while in other embodiments the direction is non-orthogonal. The opening direction 420A of the access port 420 can be orthogonal to the front-facing direction; however, in some embodiments, the direction can also be non-orthogonal. Accordingly, in some embodiments, the opening direction of one access port (e.g., the access port 420) can be greater than 90 degrees different than the opening direction of the other access port (e.g., the access port 415).

FIG. 4B shows an angled side perspective 400B, with many of the labels being the same as was illustrated in FIG. 4A. With this new perspective, however, a new strap anchor 435 is now partially visible. This strap anchor 435 is a part of the second pair of strap anchors mentioned previously. Further details on this strap anchor 435 will be provided later when it is brought into a better view.

FIG. 4B also reveals a portion of the padding 440. The padding 440 is disposed on a rear portion or rear surface of the vacuum facemask. The padding 440 is a portion of the vacuum facemask that contacts the user's face. The padding 440 can be comprised of any cushioning and sealing material, such as soft plastic, foam, neoprene, rubber, and so on.

FIG. 4B also shows a directional legend. This legend shows the top, bottom, left, right, front, and rear directions of the vacuum facemask.

FIG. 4C shows a side perspective 400C, as evidenced by the directional legend. Here, the strap anchor 435 is more visible than previously illustrated. FIG. 4C also shows the other visible components of the vacuum facemask, including the opening area of the access port 420.

FIG. 4D shows an angled rear perspective 400D. When viewed from this angle, one can observe how the padding 440 traverses and entire perimeter of the vacuum facemask and is disposed at an area where the facemask will contact the user's face. The strap anchor 435 is also now visible. Similar to the strap anchor 430, the strap anchor 435 can optionally be configured to have a rectangular shape as the opening through which the flexible strap can be wrapped around. Of course, other shapes for the opening can be used as well, such as eyelets or circles, ovals, squares, and so on.

FIG. 4E shows a rear perspective 400E of the vacuum facemask. From this vantage view, one can view the first pair of strap anchors, which includes the strap anchors 425 and 430, and a second pair of strap anchors, which includes the strap anchor 435 and the strap anchor 445. In some embodiments, the strap anchor 435 is at least partially positioned behind (relative to the front-facing direction of the facemask) the access port 420. Similarly, in some embodiments, the strap anchor 445 is at least partially positioned behind the access port 415. In some embodiments, the strap anchors 435 and 445 are positioned lower on the facemask such that they are at least partially visible when the facemask is viewed from the front.

Accordingly, in some embodiments, the vacuum facemask includes a first pair of strap anchors and a second pair of strap anchors. A first strap, which is configured to wrap around a head of the user, can be coupled to the first pair of strap anchors. Similarly, a second strap, which is also configured to wrap around the user's head, can be coupled to the second pair of strap anchors.

The size of the vacuum facemask can be configured as a “one size fits all” facemask. In some embodiments, however, the size can vary to accommodate different sized heads, such as varying sizes for children and varying sizes for adults. The width of the covering portion 405 that is provided for the user's nose can have different sizes as well to accommodate different sized noses. Similarly, the width or radius of the central lumen 410 can vary to accommodate different sized mouths. In some embodiments, the shape of the central lumen 410 is circular while in other embodiments the shape is non-circular, such as an oval shape, a square shape, a rounded edge square shape, a rectangle shape, or even a rounded edge rectangle shape.

FIG. 4F shows an angled rear perspective 400F of the vacuum facemask. From this perspective, the strap anchor 445 is more clearly visible.

FIG. 4G shows a different angled side perspective 400G. FIG. 4H shows a different angled side perspective 400H. FIG. 4I shows a flat perspective 400I, where the vacuum facemask is placed on its front end and is being viewed from a top-to-bottom perspective. FIG. 4J shows another angled side perspective 400J. With this perspective and in this embodiment, one can observe how the central lumen 410 can be a hollowed out portion of the covering portion 405. FIG. 4K shows another angled side perspective 400K. As mentioned previously, however, the central lumen can optionally include a removable cover that, when removed, permits access to the user's nose and/or mouth.

FIG. 4L shows a side perspective 400L, with the opening of the access port 415 being clearly visible. FIG. 4M shows another angled side perspective 400M.

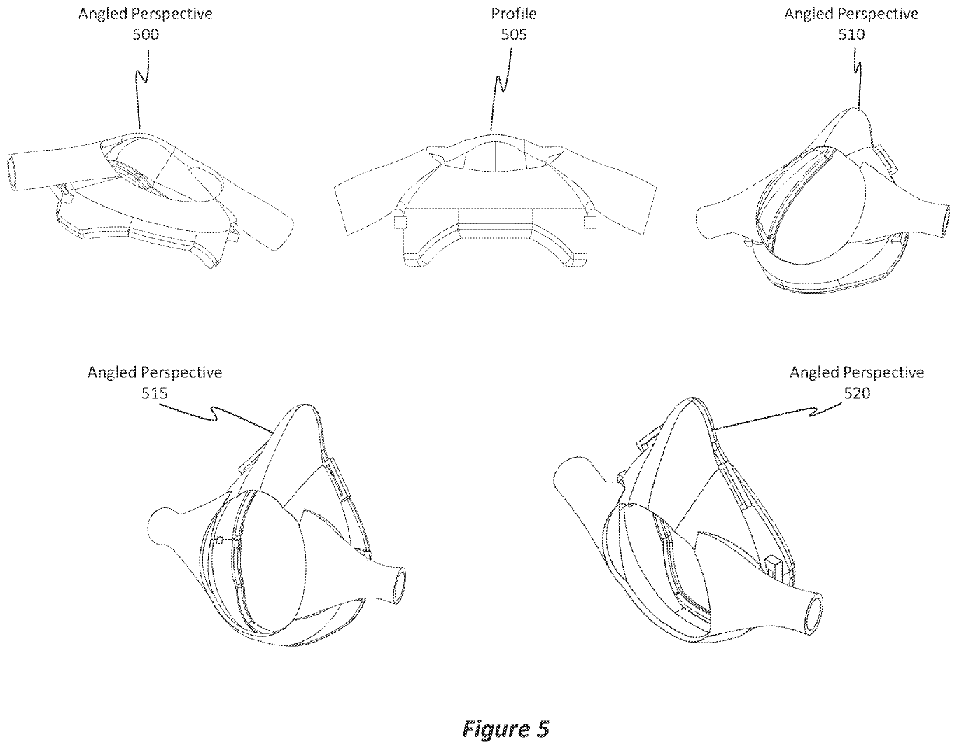

FIG. 5 shows various other perspectives and views. For instance, FIG. 5 shows an angled perspective 500, a profile 505, an angled perspective 510, an angled perspective 515, and an angled perspective 520.

Accordingly, in some embodiments, the vacuum facemask can include a covering portion that is configured to cover a portion of a face of user, a central lumen positioned in the covering portion, a first access port, and a second access port. The first and second access ports can be configured to enable communication with a vacuum.

In some cases, padding can be disposed on a rear surface of the vacuum facemask. The padding can be positioned on the vacuum facemask at a position where the vacuum facemask contacts the user's face. A first portion of a vacuum tube can connect the vacuum to the first access port, and a second portion of the vacuum tube can connect the vacuum to the second access port, as shown in FIG. 3A.

With this configuration, the vacuum can provide a sucking airflow, which sucks air away from the central lumen towards the first access port and towards the second access port. Optionally, the airflow through the first access port can be opposite the airflow through the second access port. Similarly, an opening direction of the first access port can optionally be opposite in direction to an opening direction of the second access port. Optionally, an opening direction of the first access port is at least 120 degrees different than an opening direction of the second access port. In some cases, the directions are anywhere from 90 degrees to 180 degrees different relative to one another.

In some embodiments, the padding is one of a rubber padding, a foam padding, a neoprene padding, and so forth. Beneficially, the padding seals the vacuum facemask to the user's face and substantially prevents the sucking airflow from exiting the vacuum facemask except through the first access port and the second access port. In some embodiments, the vacuum filters the sucking airflow.

In some embodiments, the vacuum facemask comprises a covering portion that is configured to cover a portion of a face of user, a central lumen positioned in the covering portion, a first access port, and a second access port. The first and second access ports are configured to enable communication with a vacuum.

The facemask can also include padding disposed on a rear surface of the vacuum facemask. The padding is positioned on the vacuum facemask at a position where the vacuum facemask contacts the user's face.

The facemask can also include a first pair of strap anchors and a first flexible strap. The first flexible strap can be coupled to the first pair of strap anchors and can be configured to wrap around a head of the user. The facemask can include a second pair of strap anchors and a second flexible strap. The second flexible strap can be coupled to the second pair of strap anchors and can be configured to wrap around the user's head.

A vacuum tube can connect the vacuum to at least one of the first access port or the second access port. The vacuum provides a sucking airflow, which sucks air away from the central lumen towards the at least one of the first access port or the second access port. Optionally, the sucking airflow sucks air only towards the second access port, such as the configuration shown in FIG. 3B. As another option, the sucking airflow sucks air towards both the first access port and the second access port, as shown by the configuration presented in FIG. 3A.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

What is claimed is:

1. A vacuum facemask comprising:
a covering portion that is configured to cover at least a nose and a mouth of a user;
a central lumen positioned in the covering portion, the central lumen being configured to permit access to the nose and/or the mouth of the user; and
an access port configured to enable airflow communication between the covering portion and a bilateral vacuum, which provides suction to generate an airflow,
wherein:
a vacuum tube connects the bilateral vacuum to the access port of the facemask, and
the airflow is directed away from the central lumen towards the bilateral vacuum via the vacuum tube to reduce or remove aerosolized particles emitted from the user's mouth or nose in an effective amount to decrease expulsion of the aerosolized particles to regions outside of the vacuum facemask.

2. The vacuum facemask of claim 1, wherein the vacuum facemask includes a second access port.

3. The vacuum facemask of claim 2, wherein the vacuum tube additionally connects the second access port to the bilateral vacuum.

4. The vacuum facemask of claim 2, wherein the access port is disposed on a first lateral position of the vacuum facemask, and wherein the second access port is disposed on a second lateral position of the vacuum facemask.

5. The vacuum facemask of claim 4, wherein an opening direction of the access port is orthogonal to a front-facing direction of the vacuum facemask.

6. The vacuum facemask of claim 5, wherein an opening direction of the second access port is greater than 90 degrees different than the opening direction of the access port.

7. The vacuum facemask of claim 1, wherein:
the vacuum facemask includes a first pair of strap anchors and a second pair of strap anchors,
a first strap, which is configured to wrap around a head of the user, is coupled to the first pair of strap anchors, and
a second strap, which is also configured to wrap around the user's head, is coupled to the second pair of strap anchors.

8. The vacuum facemask of claim 1, wherein the central lumen is a hollowed out portion of the covering portion.

9. The vacuum facemask of claim 1, wherein the central lumen includes a removable cover that, when removed, permits access to the nose and/or the mouth of the user.

10. The vacuum facemask of claim 1, wherein the vacuum facemask includes padding on a rear surface of the vacuum facemask, the padding being a portion of the vacuum facemask that contacts a face of the user.

11. A vacuum facemask comprising:
a covering portion that is configured to cover a portion of a face of user;
a central lumen positioned in the covering portion;
a first access port and a second access port, the first and second access ports being configured to enable communication with a vacuum; and
padding disposed on a rear surface of the vacuum facemask, the padding being positioned on the vacuum facemask at a position where the vacuum facemask contacts the user's face;
wherein:
a first portion of a vacuum tube connects the vacuum to the first access port,
a second portion of the vacuum tube connects the vacuum to the second access port, and
the vacuum provides a sucking airflow, which sucks air away from the central lumen towards the first access port and towards the second access port.

12. The vacuum facemask of claim 11, wherein an opening direction of the first access port is opposite in direction to an opening direction of the second access port.

13. The vacuum facemask of claim 11, wherein an opening direction of the first access port is at least 120 degrees different than an opening direction of the second access port.

14. The vacuum facemask of claim 11, wherein the padding is a rubber padding.

15. The vacuum facemask of claim 11, wherein the padding is a foam padding.

16. The vacuum facemask of claim 11, wherein the padding seals the vacuum facemask to the user's face and substantially prevents the sucking airflow from exiting the vacuum facemask except through the first access port and the second access port.

17. The vacuum facemask of claim 11, wherein the vacuum filters the sucking airflow.

18. A vacuum facemask comprising:
a covering portion that is configured to cover a portion of a face of user;
a central lumen positioned in the covering portion;
a first access port and a second access port, the first and second access ports being configured to enable communication with a vacuum;
padding disposed on a rear surface of the vacuum facemask, the padding being positioned on the vacuum facemask at a position where the vacuum facemask contacts the user's face;
a first pair of strap anchors;
a first flexible strap that is coupled to the first pair of strap anchors and that configured to wrap around a head of the user;
a second pair of strap anchors; and
a second flexible strap that is coupled to the second pair of strap anchors and that is configured to wrap around the user's head,
wherein:
a vacuum tube connects the vacuum to at least one of the first access port or the second access port, and
the vacuum provides a sucking airflow, which sucks air away from the central lumen towards the at least one of the first access port or the second access port.

19. The vacuum facemask of claim 18, wherein the sucking airflow sucks air only towards the second access port.

20. The vacuum facemask of claim 18, wherein the sucking airflow sucks air towards both the first access port and the second access port.
